(19) United States
(12) Patent Application Publication
(10) Pub. No.: US 2026/0272773 A1
(43) Pub. Date: Sep. 17, 2026

(54) ELECTRIC SCRAPING MASSAGE DEVICE

(71) Applicant: APR Co., Ltd., Seoul (KR)

(72) Inventors: Jong Chul Ji, Seoul (KR); Gyoun Jung Lee, Seoul (KR); Seung Woo Park, Seoul (KR)

(21) Appl. No.: 19/473,993

(22) PCT Filed: Apr. 25, 2024
(86) PCT No.: PCT/KR2024/005637

§ 371 Date: Oct. 9, 2025

(30) Foreign Application Priority Data
May 3, 2023 (KR) 10-2023-0057738

(51) Int. Cl. A61H 39/00; A61H 7/00; A61H 39/06

(52) U.S. Cl. CPC A61H 39/002; A61H 7/003; A61H 39/06; A61H 2230/50; A61H 2230/655

(57) ABSTRACT

The present disclosure provides an electric scraping massage device. The electric scraping massage device according to one aspect of the present disclosure may comprise a main body, a control unit, a heat generation module and an EMS generation module.

### TECHNICAL FIELD

The present disclosure relates to an electrical Gua sha massage apparatus, and more particularly, to an electrical Gua sha massage apparatus capable of selectively implementing electrical stimulation.

### BACKGROUND ART

Generally, massage refers to rubbing, tapping, or kneading the skin with hands to improve skin elasticity or relieve muscle fatigue, and may be performed using bare hands or instruments.

Gua sha massage (Gua sha therapy), which is one type of massage, is a traditional folk physiotherapy handed down from China, in which various tools are used to repeatedly press or rub along meridians and acupoints to loosen the clumped area and promote blood circulation.

A Gua sha massage apparatus for performing such Gua sha massage generally includes a handle configured to be gripped by the operator, and a protrusion provided on one side the handle in various shapes, the protrusion being configured to allow repeated pressing or rubbing of various points of the body.

Meanwhile, recently, health therapies using electrical muscle stimulation (EMS) technology have been attracting attention, and technologies have been developed to improve the efficiency of Gua sha massage by applying the EMS technology to Gua sha massage apparatuses.

For example, Korean Utility Model Publication No. 20-2022-0002575 discloses a Gua sha massage apparatus including: a housing having an upper surface and a lower surface, and four side surfaces between the upper surface and the lower surface; a low-frequency generator provided on a first side surface which is one side surface among the four side surfaces and configured to generate a low frequency wave; and a high-frequency generator provided on a second side surface which is one side surface among the four side surfaces and configured to generate a high frequency wave.

Such a Gua sha massage apparatus forms protrusions of various shapes on its outer side and is configured to perform a Gua sha massage by utilizing a stimulus generated by scraping the skin with strong pressure through the protrusions.

Meanwhile, because the muscle amount of each body portion of a subject receiving a treatment is different, and the thickness and sensitivity of the skin are different, the waveform of the EMS stimulation must be precisely designed to fit the corresponding body portion.

However, in the related art, since the waveform of the EMS stimulation was designed to have only a fixed waveform, effective electrical stimulation could not be delivered to the body portion to be treated, and rather, discomfort was often caused.

For example, there may be a portion (e.g., a non-face body portion) having a large amount of muscle and being insensitive to stimulation, and a portion (e.g., a face portion) having a small amount of muscle and being sensitive to stimulation, and as the same frequency waveform stimulation is applied to both the non-face body portion and the face portion, proper stimulation may not be obtained when performing the Gua sha massage, and rather, discomfort may occur.

On the other hand, since Gua sha massage apparatuses are known to be relatively safe, people tend to apply stronger pressure while scraping the skin using the Gua sha massage apparatus to obtain stronger stimulation.

At this time, excessive force may be applied, which may cause temporary bruises or skin discoloration such as contusions.

In addition, if the skin is sensitive or if there is an underlying disease, the Gua sha massage process may be painful and may be unsuitable for a subject having a particular medical condition.

Accordingly, there is a need for an electrical Gua sha massage apparatus capable of further enhancing the effect while compensating for drawbacks of traditional Gua sha massage through EMS waveforms designed for each body portion.

### DISCLOSURE

### Technical Problem

The present disclosure has been made to solve the above-described problems, and an object of the present disclosure is to provide an electrical Gua sha massage apparatus capable of selectively implementing electrical stimulation.

In addition, the present disclosure provides an electrical Gua sha massage apparatus that may compensate for the disadvantages of a traditional Gua sha massage while further enhancing the effect through EMS waveforms designed for each skin portion.

In addition, the present disclosure provides an electrical Gua sha massage apparatus capable of performing the function of a traditional Gua sha massage apparatus using frictional stimulation generated by scraping the skin of the body portion to be treated with strong pressure while being grasped, the electrical Gua sha massage function of adding thermal treatment by applying heat to the body portion to be treated, and the electrical Gua sha massage function of adding electrical stimulation treatment through a selective EMS waveform for each body portion to be treated.

The problems of the present disclosure are not limited to those mentioned above, and other problems not mentioned will be clearly understood by those of ordinary skill in the art from the following description.

### Technical Solution

According to an aspect of the present disclosure, an electrical Gua sha massage apparatus is provided.

The electrical Gua sha massage apparatus includes: a main body including a graspable handle and at least one skin contact configured to be frictionally in contact with a portion to be treated; a controller provided in the main body; a thermal generation module provided in the main body and configured to generate a heat under a control of the controller and to transfer the heat to the portion to be treated through the skin contact; and an EMS generation module provided in the main body and configured to generate a pulsed electrical signal of a selective waveform through the skin contact under the control of the controller according to whether the body portion to be treated is a body portion having a large amount of muscle and being insensitive to stimulation or a body portion having a small amount of muscle and being sensitive to stimulation.

Meanwhile, the electrical Gua sha massage apparatus further includes an impedance measurement module provided in the main body and configured to measure an impedance of the body portion to be treated in contact with the skin contact under control of the controller.

The electrical Gua sha massage apparatus determines, according to the impedance value measured by the impedance measurement module, whether the body portion to be treated is the body portion having a large amount of muscle and being insensitive to stimulation or the body portion having a small amount of muscle and being sensitive to stimulation and generates the pulsed electrical signal of the selective waveform through the EMS generation module.

Meanwhile, the thermal generation module may include a heater composed of a heating wire or a surface heating element.

In addition, the thermal generation module may further include a sensor configured to measure a skin temperature of the body portion to be treated in contact therewith.

Meanwhile, the EMS generation module includes a high-voltage generator; a pulse width modulation (PWM) adjuster configured to adjust a voltage generated by the high-voltage generator under the control of the controller so as to keep an amplitude of a waveform constant according to a signal to be modulated and to modulate a pulse width; and an H-bridge configured to output the voltage adjusted through the PWM adjuster as a voltage for stimulation in positive (+) and negative (−) waveforms.

Meanwhile, the main body may include a waveform selection switch provided on one side to select the waveform generated by the EMS generation module.

In addition, the impedance measurement module includes a first electrode formed by a portion of the skin contact, and a second electrode formed by another portion of the skin contact spaced apart from the first electrode.

If necessary, the impedance value measured by the impedance measurement module may be linked to an external smartphone or server through a network.

Meanwhile, the main body has a donut shape or a knuckle shape with a through-hole through which a finger of an operator passes.

In addition, the skin contact includes a first skin contact having a contact surface in which a plurality of convex protrusions and a plurality of concave grooves are arranged to have a wave shape; a second skin contact having a contact surface to be gently concave over the entire surface; and a third skin contact having a contact surface to be gently convex over the entire surface.

### Advantageous Effects

According to the above configuration, the electrical Gua sha massage apparatus according to the present disclosure may select a suitable waveform through the EMS generation module according to whether the portion to be treated is a non-face body portion having a large amount of muscle and being insensitive to stimulation or a face portion having a small amount of muscle and being sensitive to stimulation, and has the effect of further enhancing the effect of the Gua sha massage when performing the Gua sha massage.

In addition, the electrical Gua sha massage apparatus according to the present disclosure has a form similar to a traditional Gua sha massage apparatus and, since it has shapes suitable for stimulation for each body portion, it has the effect of maximizing the efficiency of the Gua sha massage by achieving a high degree of adherence to the skin despite bending of various body portions.

In addition, through the thermal generation module, thermotherapy is enabled, and as the skin tissue temperature rises due to the thermal function, capillary dilation occurs in the skin, blood flow is increased, lymphatic circulation is promoted, and metabolism is enhanced, so that, together with EMS stimulation of the EMS generation module, the effect of the Gua sha massage may be further enhanced.

In addition, through the impedance measurement module, an EMS waveform is automatically selected for the body portion to be treated in contact with the skin contact, so that an effective Gua sha massage treatment may be performed and user satisfaction with the Gua sha massage apparatus may be improved.

In addition, since the thermal function using the thermal generation module and the EMS stimulation function using the EMS generation module may be selectively used under the control of the controller, it has the effect of treating a body portion to be treated where pain is strongly perceived so that the degree of pain is reduced and the painful body portion is improved.

In addition, since the EMS stimulation using the EMS generation module is selectively performed for each body portion, the body portion to be treated may be protected and a safe Gua sha massage treatment may be performed.

In addition, it has the effect of performing, together, the function of a traditional Gua sha massage apparatus using frictional stimulation generated by scraping the skin of the body portion to be treated with strong pressure while being grasped, the electrical Gua sha massage function of adding thermal treatment by applying heat to the body portion to be treated, and the electrical Gua sha massage function of adding electrical stimulation treatment through a selective EMS waveform for each body portion to be treated.

Advantageous effects of the present disclosure are not limited to the above-described effects and should be understood to include all effects that can be inferred from the configuration of the invention described in the detailed description or claims of the present disclosure.

### DESCRIPTION OF THE DRAWINGS

FIG. 1 is a view illustrating an electrical Gua sha massage apparatus according to an embodiment of the present disclosure.

FIG. 2 is a configuration diagram illustrating the electrical Gua sha massage apparatus according to an embodiment of the present disclosure.

FIG. 3 is a configuration diagram illustrating a thermal generation module applied to an electrical Gua sha massage apparatus according to an embodiment of the present disclosure.

FIG. 4 is a schematic view illustrating a heater of the thermal generation module applied to the electrical Gua sha massage apparatus according to an embodiment of the present disclosure.

FIG. 5 is a schematic configuration diagram illustrating an EMS generation module applied to an electrical Gua sha massage apparatus according to an embodiment of the present disclosure.

FIG. 6 is a diagram illustrating an example of a circuit diagram of the EMS generation module of FIG. 5.

FIG. 7 is a waveform diagram illustrating an EMS stimulation function for a face portion through the EMS generation module applied to the electrical Gua sha massage apparatus according to an embodiment of the present disclosure.

FIG. 8 is a waveform diagram illustrating an EMS stimulation function for a non-face body portion through the EMS generation module applied to the electrical Gua sha massage apparatus according to an embodiment of the present disclosure.

FIG. 9 is a schematic configuration diagram illustrating an impedance measurement module applied to an electrical Gua sha massage apparatus according to an embodiment of the present disclosure.

FIG. 10 is a schematic view illustrating electrodes provided at the skin contact to configure the impedance measurement module applied to the electrical Gua sha massage apparatus according to an embodiment of the present disclosure.

FIG. 11 is a diagram illustrating various skin contacts for performing a Gua sha massage treatment of the electrical Gua sha massage apparatus according to an embodiment of the present disclosure.

### BEST EMBODIMENTS

The present disclosure provides, in its best form, an electrical Gua sha massage apparatus, comprising: a main body including a graspable handle and at least one skin contact configured to be frictionally in contact with a portion to be treated; a controller provided in the main body; a thermal generation module provided in the main body and configured to generate a heat under a control of the controller and to transfer the heat to the portion to be treated through the skin contact; and an EMS generation module provided in the main body and configured to generate a pulsed electrical signal of a selective waveform through the skin contact under the control of the controller according to whether the body portion to be treated is a body portion having a large amount of muscle and being insensitive to stimulation or a body portion having a small amount of muscle and being sensitive to stimulation.

### Detailed Description of the Embodiments

Hereinafter, exemplary embodiments of the present disclosure will be described in detail so that those of ordinary skill in the art can readily implement the present disclosure with reference to the accompanying drawings. The present disclosure may be embodied in many different forms and is not limited to the embodiments set forth herein. In the drawings, parts unrelated to the description are omitted for clarity in describing the present disclosure, and throughout the specification, the same or similar reference numerals denote the same or similar elements.

The words and terms used in the present specification and claims should not be construed as being limited to their ordinary or dictionary meanings but should be construed as having meanings and concepts consistent with the technical spirit of the present disclosure, in accordance with the principle that an inventor may define terms and concepts to best describe the present disclosure.

Accordingly, the embodiments described in the present specification and the configurations shown in the drawings correspond to preferred embodiments of the present disclosure, and do not represent all the technical idea of the present disclosure, so the configurations may have various examples of equivalent and modification that can replace them at the time of filing the present disclosure.

It should be understood that the terms “comprise” or “include” or “have” and the like, when used in the present specification, are intended to specify the presence of stated features, numbers, steps, operations, elements, components, or combinations thereof, but do not preclude the possibility of the presence or addition of one or more other features, numbers, steps, operations, elements, components, or combinations thereof.

The presence of an element in/on “front”, “rear”, “upper or above or top” or “lower or below or bottom” of another element includes not only being disposed in/on “front”, “rear”, “upper or above or top” or “lower or below or bottom” directly in contact with other elements, but also cases in which another element being disposed in the middle, unless otherwise specified. In addition, unless otherwise specified, the expression that an element is “connected” to another element should be construed to include not only a case of being directly connected to each other but also a case of being indirectly connected to each other.

In addition, in describing the present disclosure, detailed descriptions of related known functions or configurations will be omitted so as not to obscure the gist of the present disclosure.

Hereinafter, an electrical Gua sha massage apparatus according to an embodiment of the present disclosure will be described with reference to the drawings.

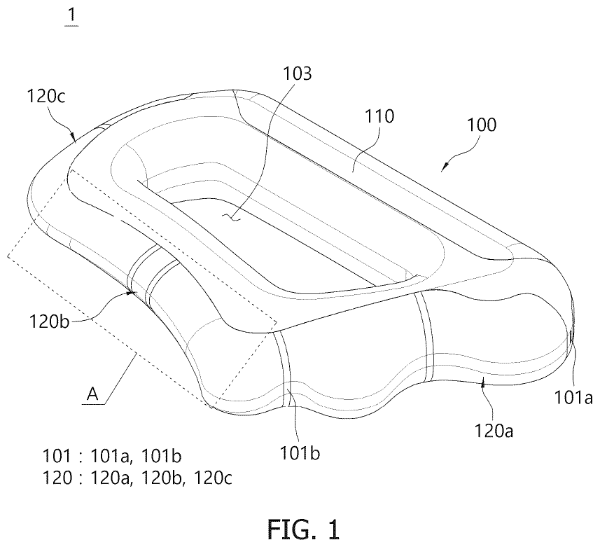

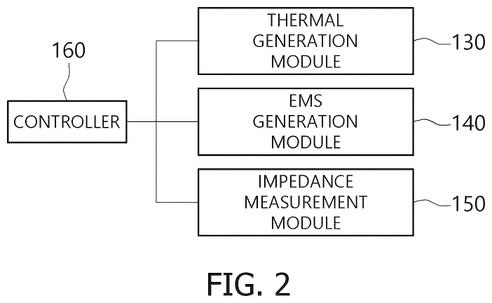

FIG. 1 is a view illustrating an electrical Gua sha massage apparatus according to an embodiment of the present disclosure, and FIG. 2 is a configuration diagram illustrating the electrical Gua sha massage apparatus according to an embodiment of the present disclosure.

As illustrated, the electrical Gua sha massage apparatus 1 according to an embodiment of the present disclosure allows a Gua sha massage treatment to be performed more safely while protecting the skin of a body portion to be treated during the process of performing the Gua sha massage treatment.

In addition, by performing a thermal function on a body portion to be treated through the thermal generation module 130 and selectively performing an EMS stimulation function on a body portion to be treated through the EMS generation module 140, the massage effect during the Gua sha massage treatment may be further maximized.

Meanwhile, the EMS generation module 140 may select a suitable waveform according to whether the portion to be treated is a non-face body portion having a large amount of muscle and being insensitive to stimulation or a face portion having a small amount of muscle and being sensitive to stimulation, and thus may further enhance the effect of the Gua sha massage when performing the Gua sha massage.

In a face portion having a relatively small amount of muscle and being sensitive to stimulation, a short pulse width may be formed by using a frequency higher than that applied to a non-face body portion, thereby enhancing the effect of the Gua sha massage using electrical stimulation.

To this end, the electrical Gua sha massage apparatus 1 according to an embodiment of the present disclosure may select an EMS electrical stimulation waveform of the EMS generation module 140 according to the operator's selection by using the selection switch 102 provided on one side of the main body 100.

As another example, the electrical Gua sha massage apparatus 1 according to an embodiment of the present disclosure may further include an impedance measurement module 150 configured to measure an impedance of the body portion to be treated in contact with the skin contact 120 under the control of the controller 160, and may select an EMS waveform of the EMS generation module 140 according to the measured impedance value as well.

Such an electrical Gua sha massage apparatus 1 according to an embodiment of the present disclosure is provided with a controller 160, a thermal generation module 130, an EMS generation module 140, and an impedance measurement module 150 inside the main body 100.

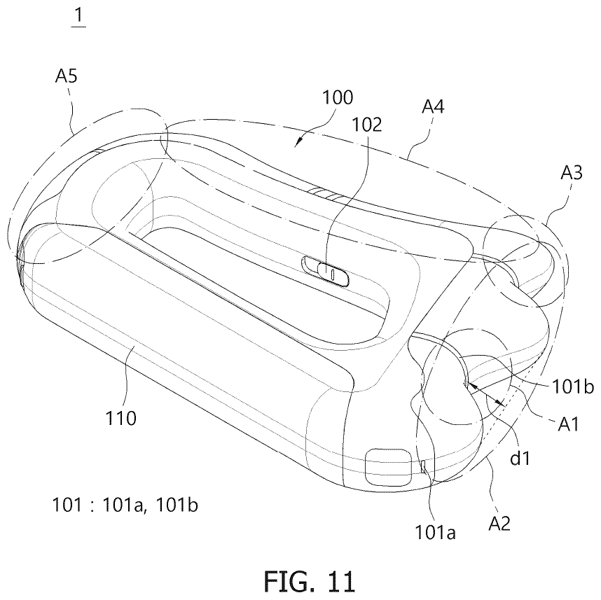

FIG. 11 is a diagram illustrating various skin contacts for performing a Gua sha massage treatment of the electrical Gua sha massage apparatus according to an embodiment of the present disclosure. Referring to FIGS. 1 and 2 together, the electrical Gua sha massage apparatus 1 according to the above-described embodiment of the present disclosure will be described in more detail below.

First, the portion to be treated refers to a body portion such as the face, hand, arm, or leg where a Gua sha massage may be performed by applying pressure to generate friction.

In addition, the operator refers to a person who holds the electrical Gua sha massage apparatus 1 and performs the Gua sha massage, and the subject refers to a person who receives the Gua sha massage from the operator.

In this case, the operator and the subject may be the same person or different persons.

The electrical Gua sha massage apparatus 1 according to an embodiment of the present disclosure includes a main body 100 configured to generate friction by applying pressure while being in contact with the skin of a body portion to be treated.

In this case, the main body 100 largely includes a handle 110 and a skin contact 120.

The handle 110 provided in the main body 100 may have a bar or rod shape graspable by the operator.

In addition, the skin contact 120 has a structure in which a bar or rod shape extends from or is connected to the handle 110, and at least one contact surface is configured to be frictionally in contact with the skin of the body portion to be treated.

Such a skin contact 120 may be formed at various portions of the main body 100 according to the shape of the main body 100, and the main body 100 includes at least one such skin contact 120.

Meanwhile, for example, the main body 100 including the above-described handle 110 and skin contact 120 may have a donut shape with a through-hole 103 through which a finger of the operator passes (see FIG. 1). Alternatively, in another example, the main body 100 may have a knuckle shape in which a plurality of through-holes 103 are formed to allow each finger of the operator to pass through.

The main body 100 constituting such an electrical Gua sha massage apparatus 1 is not limited to any one shape as long as it has a shape graspable by the operator, and various shapes may be applied as long as it can generate friction by applying pressure to the skin of the subject.

In addition, such a main body 100 may be made of various materials such as synthetic resin, metal, or stone as needed.

Meanwhile, in an embodiment for explaining the present disclosure, the main body 100 of the electrical Gua sha massage apparatus 1 is illustrated and described as having a quadrangular donut shape with a through-hole 103 and four side surfaces including a top, bottom, left, and right (see FIG. 1).

In this case, the handle 110 forms an upper portion of the main body 100 and has a graspable rod shape.

And the skin contact 120 forms at least one contact surface 121 that is in contact with the skin and is disposed at the left side, right side, and bottom of the above-described handle 110.

Accordingly, the skin contact 120 has respective contact surfaces 121 and is composed of a first skin contact 120a, a second skin contact 120b, and a third skin contact 120c.

Meanwhile, the contact surfaces 121 of the first skin contact 120a, the second skin contact 120b, and the third skin contact 120c may be configured to form protruding portions of different shapes, and the operator may perform the Gua sha massage by selecting the skin contact 120 having contact surfaces 121 of various shapes according to the body portion to be treated.

Referring to FIGS. 1 and 11 together, the first skin contact 120a may form a contact surface in which a plurality of convex protrusions and a plurality of concave grooves are arranged to have a wave shape.

Again, the first skin contact 120a forms a V zone A1 capable of massaging a jaw portion of the body portion to be treated by using a concave groove that has a gap dl between convex protrusions and is formed in a deep groove shape.

In addition, the first skin contact 120a forms a medicinal hand fist zone A2 capable of massaging a stiff muscle portion, the neck, the axilla, and the like by using the wave shape formed by the plurality of convex protrusions and concave grooves.

In addition, the first skin contact 120a forms an acupressure zone A3 capable of massaging the eye and eyebrow portions by using the last protruding convex protrusion.

Meanwhile, the second skin contact 120b may form a curved contact surface to be gently concave over the entire surface.

Using this, the second skin contact 120b forms a concave medicinal hand edge zone A4 capable of massaging a convex portion of a body portion to be treated.

In addition, the third skin contact 120c may form a contact surface to be gently convex over the entire surface.

Using this, the third skin contact 120c forms a convex medicinal hand base zone A5 capable of massaging a concave portion of the body portion to be treated.

As described above, the electrical Gua sha massage apparatus 1 according to an embodiment of the present disclosure is configured such that the skin contact 120 has different protruding portions or concave portions suitable for stimulation of each body portion to be treated, and even in the bending of various portions of the body, it has a high degree of adherence to the skin of the body portion to be treated, thereby maximizing the efficiency of the Gua sha massage.

Meanwhile, the contact surface of the skin contact 120 is preferably formed of a material that does not cause a wound to the skin of the body portion to be treated while being in contact with the skin of the body portion to be treated and generating friction due to applied pressure.

For example, the contact surface 121 may be formed at an end of the skin contact 120 using ceramic, silver, wood, red agate, jade, plastic materials, or the like.

The skin contact 120 generates friction by being in contact with the skin of the body portion to be treated and moving along the portion to be treated according to the applied pressure.

Meanwhile, as described above, the first skin contact 120a, the second skin contact 120b, and the third skin contact 120c constituting the skin contact 120 transmit, as an electrical stimulus, a pulsed electrical signal of a predetermined frequency band generated by the electrical muscle stimulation (EMS) generation module 140 provided in the main body 100 to the body portion to be treated where the Gua sha massage is performed.

In addition, the thermal generation module 130 provided in the main body 100 may perform thermal therapy on the body portion to be treated where the Gua sha massage is performed by using heat generated therefrom and may enhance the effect of the Gua sha massage by assisting blood circulation.

Referring again to FIGS. 1 and 2, the electrical Gua sha massage apparatus 1 according to an embodiment of the present disclosure includes a main body 100, and the main body 100 includes a controller 160, a thermal generation module 130, an EMS generation module 140, and, if necessary, an impedance measurement module 150.

As described above, the main body 100 has a graspable handle 110 and at least one skin contact 120 configured to be frictionally in contact with the body portion to be treated.

The controller 160 is provided in the main body 100 and controls the operation of the thermal generation module 130, the EMS generation module 140, and the impedance measurement module 150.

The thermal generation module 130 is provided in the main body 100 and generates heat under the control of the controller 160 to transfer the heat to the skin contact 120.

The EMS generation module 140 is provided in the main body 100 and generates a pulsed electrical signal of a selective waveform through the skin contact 120 under the control of the controller 160 according to whether the body portion to be treated has a large amount of muscle and is insensitive to stimulation or has a small amount of muscle and is sensitive to stimulation.

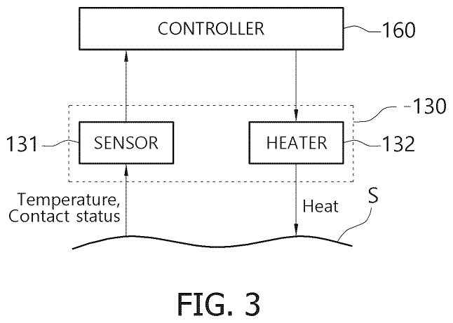

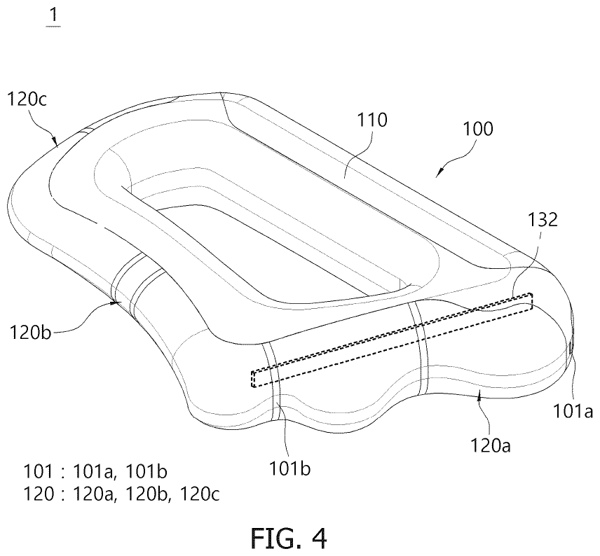

FIG. 3 is a schematic configuration diagram illustrating a thermal generation module applied to an electrical Gua sha massage apparatus according to an embodiment of the present disclosure, and FIG. 4 is a schematic view illustrating a heater of the thermal generation module applied to the electrical Gua sha massage apparatus according to an embodiment of the present disclosure.

The thermal generation module 130 applied to the electrical Gua sha massage apparatus 1 according to an embodiment of the present disclosure constitutes a heater 132 by using a heating wire or a surface heating element.

For example, referring to FIG. 4, the heater 132 is disposed at the first skin contact 120a side and, under the control of the controller 160, generates and transfers heat to the first skin contact 120a.

The first skin contact 120a, to which heat is transferred through the heater 132, is brought into contact with the body portion to be treated where the Gua sha massage is performed, and heat is transferred to the skin, thereby enabling thermal therapy.

Through this, the electrical Gua sha massage apparatus 1 according to an embodiment of the present disclosure helps blood circulation and enhances the effect of the Gua sha massage.

Meanwhile, the thermal function through the thermal generation module 130 increases the skin tissue temperature of a body portion to be treated where the Gua sha massage is performed, and causes capillary dilation in the skin, increases blood flow, promotes lymphatic circulation, and enhances metabolism.

This may further enhance the efficiency of the Gua sha massage by adding a synergistic effect to the physical friction stimulation of the Gua sha massage together with the EMS stimulation described below.

Preferably, the heater 132 is not limited to the heating wire or the surface heating element, and may also be a PTC element, a carbon element, a nichrome wire, or another type of heating element.

Further, the heat generated through the thermal generation module 130 is preferably higher than the skin temperature of the body portion to be treated, and may be 40 to 50° C.

Meanwhile, the thermal generation module 130 may further include a sensor 131 to check the temperature of the body portion to be treated as needed. The sensor 131 may be of a contact type or a non-contact type and measures the skin temperature of the body portion to be treated through the skin contact 120 in contact with the body portion and informs the controller 160 of the measured skin temperature.

The sensor 131 may be a temperature sensor that includes a contact sensor.

In other words, the sensor 131 may measure a temperature of the body portion to be treated in contact with the skin contact 120 or may identify whether the skin contact 120 is in contact with the skin or is spaced apart from the skin.

Accordingly, the electrical Gua sha massage apparatus 1 according to an embodiment of the present disclosure may be brought into contact with the body portion to be treated in a state where the heater 132 has generated heat in advance under the control of the controller 160, that is, in a state where the skin contact 120 already has a predetermined temperature, or may generate heat by operating the heater 132 in a state where the skin is brought into contact through the sensor 131.

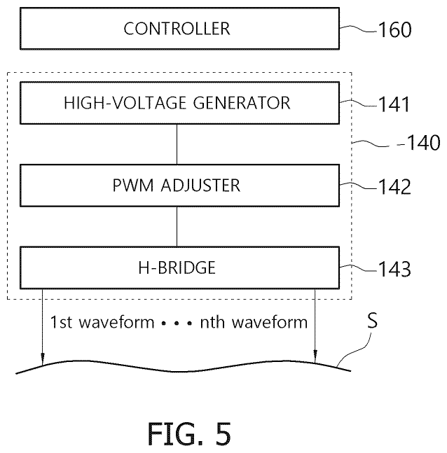

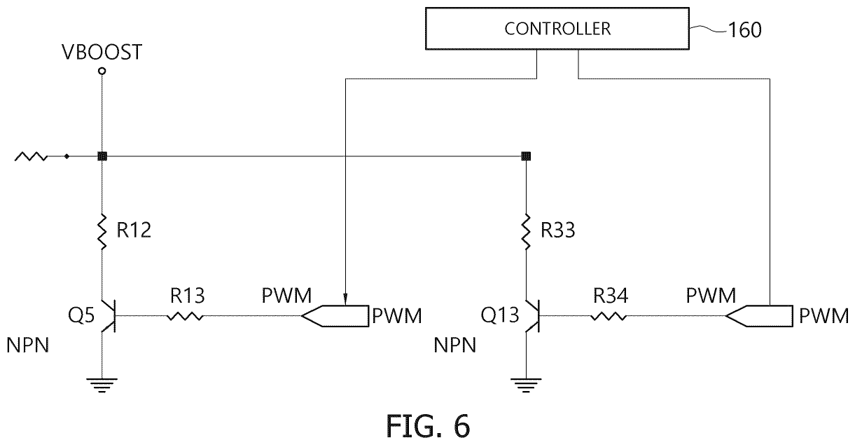

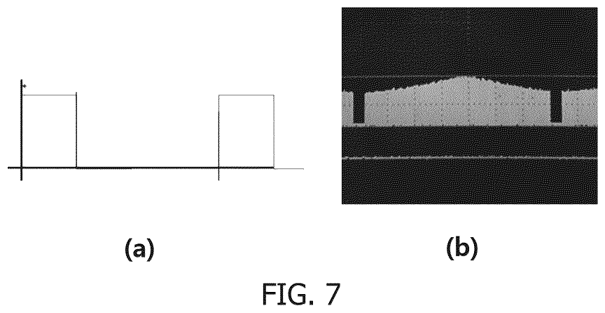

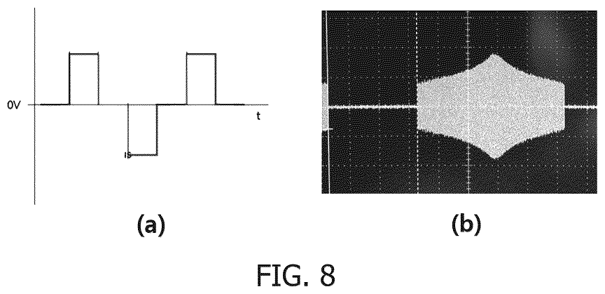

Meanwhile, FIG. 5 is a schematic configuration diagram illustrating an EMS generation module applied to an electrical Gua sha massage apparatus according to an embodiment of the present disclosure, FIG. 6 is a diagram illustrating an example of a circuit diagram of the EMS generation module of FIG. 5, FIG. 7 is a waveform diagram illustrating an EMS stimulation function for a face portion through the EMS generation module applied to the electrical Gua sha massage apparatus according to an embodiment of the present disclosure, and FIG. 8 is a waveform diagram illustrating an EMS stimulation function for a non-face body portion through the EMS generation module applied to the electrical Gua sha massage apparatus according to an embodiment of the present disclosure.

As described above, the electrical Gua sha massage apparatus 1 according to an embodiment of the present disclosure is configured to select an appropriate waveform through the EMS generation module 140 according to whether the portion to be treated is a non-face body portion having a large amount of muscle and being insensitive to stimulation, or a face portion having a small amount of muscle and being sensitive to stimulation.

The selection of the waveform may be directly made by the operator through a waveform selection switch 102 (FIG. 11) provided at one side of the main body 100.

In addition, in the electrical Gua sha massage apparatus 1 according to an embodiment of the present disclosure, the pulsed electrical signal generated by the EMS generation module 140 is described as being divided into two types: one for the non-face body portion having a large amount of muscle and being insensitive to stimulation, and the other for the face portion having a small amount of muscle and being sensitive to stimulation. However, such waveforms may be stored in the controller 160 for each body portion and may also be different pulsed electrical signals for the hand, foot, leg, body, face, and the like as needed.

To this end, the EMS generation module 140 provided in the main body 100 of the electrical Gua sha massage apparatus 1 according to an embodiment of the present disclosure generates a pulsed electrical signal of a selective waveform under the control of the controller 160.

This pulsed electrical signal is transmitted through the skin contact 120 to the body portion to be treated where the Gua sha massage is performed with electrical stimulation.

Referring to FIG. 5, the EMS generation module 140 provided in the electrical Gua sha massage apparatus 1 according to an embodiment of the present disclosure includes a high-voltage generator 141, a pulse width modulation (PWM) adjuster 142 configured to adjust a voltage generated by the high-voltage generator 141 under the control of the controller 160 so as to keep an amplitude of a waveform constant according to a signal to be modulated and to modulate a pulse width, and an H-bridge 143 configured to output the voltage adjusted through the PWM adjuster 142 as a voltage for stimulation in positive (+) and negative (−) waveforms.

Referring to FIG. 6 together with FIG. 5, the EMS generation module 140 provided in the electrical Gua sha massage apparatus 1 according to an embodiment of the present disclosure generates a pulsed electrical signal of a waveform suitable for a non-face body portion having a large amount of muscle and being insensitive to stimulation, and for a face portion having a small amount of muscle and being sensitive to stimulation.

In other words, the high-voltage generator 141 of the EMS generation module 140, generates a high voltage through a booster circuit for voltage boosting, and if it is determined through the waveform selection switch 102 (FIG. 11) or the impedance measurement module 150 to be described below which waveform of a pulsed electrical signal is to be generated, the PWM adjuster 142 and the H-bridge 143, under the control signal of the controller 160, generate a pulsed electrical signal of a waveform suitable for a non-face body portion having a large amount of muscle and being insensitive to stimulation, or generate a pulsed electrical signal of a waveform suitable for a face portion having a small amount of muscle and being sensitive to stimulation.

Meanwhile, preferably, referring to FIGS. 7 and 8, a waveform for EMS stimulation (electrical stimulation) generated by the EMS generation module 140 provided in the electrical Gua sha massage apparatus 1 according to an embodiment of the present disclosure may be varied in intensity in three or more steps as needed.

FIG. 7 is a waveform diagram illustrating a waveform for an EMS stimulation function of a face portion through the EMS generation module 140 according to an embodiment of the present disclosure, wherein part (a) of FIG. 7 illustrates a basic waveform, and part (b) of FIG. 7 illustrates an example of a three-step waveform for the face portion.

FIG. 8 is a waveform diagram illustrating a waveform for an EMS stimulation function of a non-face body portion through the EMS generation module 140 according to an embodiment of the present disclosure, wherein part (a) of FIG. 8 illustrates a basic waveform, and part (b) of FIG. 8 illustrates an example of a three-step waveform for the non-face body portion.

As described above, the waveform applied through the EMS generation module 140 provided in the electrical Gua sha massage apparatus 1 according to an embodiment of the present disclosure is designed so that a subject receiving the Gua sha massage does not feel discomfort and is configured as a non-linear exponential incremental wave to provide a clear stimulation.

In addition, the contact surface of the skin contact 120 serves as an electrode, and since a specific electrode is not fixedly allocated to a specific waveform, all the stimulation zones A1 to A5 having different configurations provided for Gua sha massage of the face and the non-face body portion may be smoothly utilized.

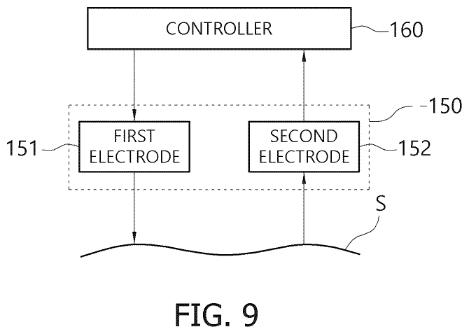

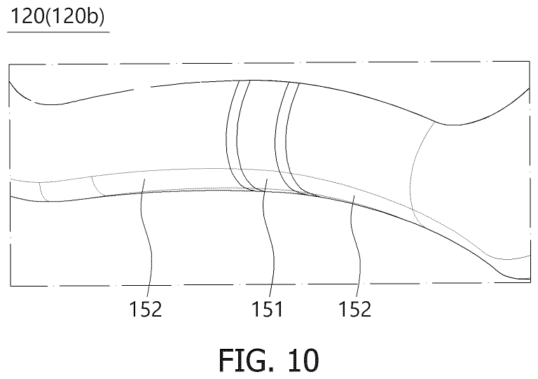

Meanwhile, FIG. 9 is a schematic configuration diagram illustrating an impedance measurement module applied to an electrical Gua sha massage apparatus according to an embodiment of the present disclosure, and FIG. 10 is a schematic view illustrating electrodes provided at the skin contact to configure the impedance measurement module applied to the electrical Gua sha massage apparatus according to an embodiment of the present disclosure.

As described above, the electrical Gua sha massage apparatus 1 according to an embodiment of the present disclosure generates, through the skin contact 120, a pulsed electrical signal of a selective waveform according to whether the body portion has a large amount of muscle and is insensitive to stimulation or a body portion has a small amount of muscle and is sensitive to stimulation, and applies electrical stimulation to the skin.

In this case, the waveform selection of the EMS generation module 140 may be a manual selection made by the operator using the waveform selection switch 102 or may be an automatic selection based on a skin impedance value measured by the impedance measurement module 150.

In other words, the electrical Gua sha massage apparatus 1 according to an embodiment of the present disclosure further includes an impedance measurement module 150 provided in the main body 100 and configured to measure an impedance of the body portion to be treated in contact with the skin contact 120 under the control of the controller 160.

It is determined, according to the impedance value measured through the impedance measurement module 150, whether the body portion to be treated where the skin contact 120 is currently performing the Gua sha massage is a body portion having a large amount of muscle and being insensitive to stimulation, or a body portion having a small amount of muscle and being sensitive to stimulation.

Accordingly, the controller 160 generates a pulsed electrical signal of a selective waveform through the EMS generation module 140 described above.

To this end, referring to FIG. 10, the impedance measurement module 150 is configured such that a portion of the skin contact 120 serves as a first electrode 151, and another portion of the skin contact 120 spaced apart from the first electrode 151 serves as a second electrode 152.

Based on the impedance measured through both ends of the impedance electrodes, that is, the first electrode 151 and the second electrode 152, it is determined which body portion to be treated is currently subjected to the Gua sha massage through the electrical Gua sha massage apparatus 1.

In other words, under the control of the controller 160, the impedance measurement module 150 measures an impedance by flowing a current through the first electrode 151 and the second electrode 152. The measured impedance value is input to the controller 160, and the controller 160 determines which body portion to be treated it is according to the impedance value. Then, according to the determined body portion to be treated, the EMS generation module 140 generates an electrical signal and applies electrical stimulation with a waveform suitable for the body portion to be treated.

Meanwhile, the impedance value measured by the impedance measurement module 150 may be linked to an external smartphone or server through a network.

In other words, impedance values for each body portion may be stored according to a subject receiving the treatment by using the electrical Gua sha massage apparatus 1, which enables the Gua sha massage treatment to be performed with electrical stimulation appropriate for each subject.

As described above, the electrical Gua sha massage apparatus 1 according to an embodiment of the present disclosure may select a suitable waveform through the EMS generation module 140 according to whether the portion to be treated is a non-face body portion having a large amount of muscle and being insensitive to stimulation or a face portion having a small amount of muscle and being sensitive to stimulation, and thus may further enhance the effect of the Gua sha massage when performing the Gua sha massage.

In addition, the electrical Gua sha massage apparatus 1 according to an embodiment of the present disclosure has a main body 100 in a form similar to a traditional Gua sha massage apparatus, and the massage zones A1 to A5 formed by the first skin contact 120a, the second skin contact 120b, and the third skin contact 120c have shapes suitable for stimulation for each body portion, so that, despite bending of various body portions, a high degree of adherence to the skin may be achieved to maximize the efficiency of the Gua sha massage.

Furthermore, through the thermal generation module 130, thermotherapy is enabled, and as the skin tissue temperature rises due to the thermal function, capillary dilation occurs in the skin, blood flow is increased, lymphatic circulation is promoted, and metabolism is enhanced, so that, together with EMS stimulation of the EMS generation module 140, the effect of the Gua sha massage may be further enhanced.

Meanwhile, through the impedance measurement module 150, an EMS waveform is automatically selected for the body portion to be treated in contact with the skin contact 120, so that an effective Gua sha massage treatment may be performed and user satisfaction with the Gua sha massage apparatus may be improved.

In addition, since the thermal function using the thermal generation module 130 and the EMS stimulation function using the EMS generation module 140 may be selectively used under the control of the controller, a body portion to be treated where pain is strongly perceived may be treated so that the degree of pain may be reduced, and the painful body portion may be improved.

And, since the EMS stimulation using the EMS generation module 140 is selectively performed for each body portion, the body portion to be treated may be protected and a safe Gua sha massage treatment may be performed.

In addition, by grasping the main body 100, the apparatus may perform, together, the function of a traditional Gua sha massage apparatus using frictional stimulation generated by scraping the skin of the body portion to be treated with strong pressure, the electrical Gua sha massage function of adding thermal treatment by applying heat to the body portion to be treated, and the electrical Gua sha massage function of adding electrical stimulation treatment through a selective EMS waveform for each body portion to be treated.

Reference numeral 101, which is not described, is an operation state LED and may be classified into a functional state LED 101b illustrating whether the thermal function or the EMS stimulation function is being performed, and an entire operation state LED 101a illustrating whether power is applied to the electrical Gua sha massage apparatus 1.

Although exemplary embodiments of the present disclosure have been described, the spirit of the present disclosure is not limited to the embodiments set forth herein. Those of ordinary skill in the art who understand the spirit of the present disclosure may easily propose other embodiments through supplement, change, removal, addition, etc. of elements within the same scope of spirit, but the embodiments will be also within the scope of the present disclosure.

1. An electrical Gua sha massage apparatus comprising:
a main body including a graspable handle and at least one skin contact configured to be frictionally in contact with a portion to be treated;
a controller provided in the main body;
a thermal generation module provided in the main body and configured to generate a heat under a control of the controller and to transfer the heat to the portion to be treated through the skin contact; and
an EMS generation module provided in the main body and configured to generate a pulsed electrical signal of a selective waveform through the skin contact under the control of the controller according to whether the body portion to be treated is a body portion having a large amount of muscle and being insensitive to stimulation or a body portion having a small amount of muscle and being sensitive to stimulation.

2. The electrical Gua sha massage apparatus of claim 1, further comprising:
an impedance measurement module provided in the main body and configured to measure an impedance of the body portion to be treated in contact with the skin contact under control of the controller,
wherein the electrical Gua sha massage apparatus determines, according to the impedance value measured by the impedance measurement module, whether the body portion to be treated is the body portion having a large amount of muscle and being insensitive to stimulation or the body portion having a small amount of muscle and being sensitive to stimulation and generates the pulsed electrical signal of the selective waveform through the EMS generation module.

3. The electrical Gua sha massage apparatus of claim 1, wherein the thermal generation module includes a heater composed of a heating wire or a surface heating element.

4. The electrical Gua sha massage apparatus of claim 3, wherein the thermal generation module further includes a sensor configured to measure a skin temperature of the body portion to be treated in contact therewith.

5. The electrical Gua sha massage apparatus of claim 1, wherein the EMS generation module includes:
a high-voltage generator;
a pulse width modulation (PWM) adjuster configured to adjust a voltage generated by the high-voltage generator under the control of the controller so as to keep an amplitude of a waveform constant according to a signal to be modulated and to modulate a pulse width; and
an H-bridge configured to output the voltage adjusted through the PWM adjuster as a voltage for stimulation in positive and negative (−) waveforms.

6. The electrical Gua sha massage apparatus of claim 1, wherein the main body includes a waveform selection switch provided on one side to select the waveform generated by the EMS generation module.

7.The electrical Gua sha massage apparatus of claim 2, wherein the impedance measurement module includes:
a first electrode formed by a portion of the skin contact, and
a second electrode formed by another portion of the skin contact spaced apart from the first electrode.

8. The electrical Gua sha massage apparatus of claim 2, wherein the impedance value measured by the impedance measurement module is linked to an external smartphone or server through a network.

9. The electrical Gua sha massage apparatus of claim 1, wherein the main body has a donut shape or a knuckle shape with a through-hole through which a finger of an operator passes.

10. The electrical Gua sha massage apparatus of claim 9, wherein the skin contact includes:
a first skin contact having a contact surface in which a plurality of convex protrusions and a plurality of concave grooves are arranged to have a wave shape;
a second skin contact having a contact surface to be gently concave over the entire surface; and
a third skin contact having a contact surface to be gently convex over the entire surface.
